(19) United States
(12) Patent Application Publication
(10) Pub. No.: US 2012/0107775 A1
(43) Pub. Date: May 3, 2012

(54) Methods of Using Botanical-Based Compositions to Treat Microbial Dental Disease

(76) Inventor: Robert M. Block, Leesburg, VA (US)

(21) Appl. No.: 13/149,071

(22) Filed: May 31, 2011

Related U.S. Application Data
(60) Provisional application No. 61/349,433, filed on May 28, 2010.

(51) Int. Cl. A61C 5/04; A61K 36/00; A61K 31/4355; A61P 31/00; A61C 17/20; A61N 5/067

(52) U.S. Cl. 433/224; 514/280; 424/725

(57) ABSTRACT

The present invention relates to methods of using botanical-based compositions to treat or prevent microbial dental disease. The claimed invention is directed to methods of using botanical-based compositions in a rinse, gel- or paste-like form to prevent microbe invasion of dental cavities, including the root canal.

### INCORPORATION BY REFERENCE

The present application claims priority from Provisional U.S. Patent Application No. 61/349,433 filed on May 28, 2010, the disclosure of which is incorporated herein by reference in its entirety.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention is directed to methods of using botanical-based compositions to treat or prevent microbial dental disease. In particular, the claimed invention relates to methods of using botanical-based irrigants to prevent microbe invasion of dental cavities, including the root canal.

### BACKGROUND OF THE INVENTION

Dentin is a calcified tissue of the body, and along with enamel, cementum, and pulp is one of the four major components of teeth. Usually, it is covered by enamel on the crown and cementum on the root and surrounds the entire pulp. By weight, seventy percent of dentin consists of the mineral hydroxylapatite, twenty percent is organic material and ten percent is water [Cate, A. R. Ten. Oral Histology: development, structure, and function. 5th ed. 1998. p. 150]. Dentin consists of microscopic channels, called dentinal tubules, which radiate outward through the dentin from the pulp to the exterior cementum or enamel border [Ross, Michael H., Gordon I. Kaye. and. Wojciech Pawlina, 2003. Histology: a text and atlas. 4th edition. Page 450]. These tubules contain fluid and cellular structures. As a result, dentin has a degree of permeability, which can increase the sensation of pain and the rate of tooth decay and/or disease.

The formation of dentin, known as dentinogenesis, begins prior to the formation of enamel and is initiated by the odontoblasts of the pulp. Unlike enamel, dentin continues to form throughout life and can be initiated in response to stimuli, such as tooth decay or attrition. There are different types of dentin, differentiated by appearance and stage of development.

Primary dentin forms most of the tooth. Secondary dentin develops after root formation is complete and forms much more slowly than primary dentin. Tertiary dentin forms as a biological response to stimuli.

Bacterial penetration of dentin and enamel leads to caries, a progressive destruction of the bone structure, and eventually pulpal inflammation and periapical abscesses, which form at the tip of the root of a tooth.

Original studies on germ free rats demonstrated that without bacterial caries, pulpal and periapical diseases do not occur [Bacteriological Studies on Experimental Dental Caries in the Rat: I. Bacterial Flora of Normal. Non-Carious Teeth. R. W. Harrison. The Journal of Infectious Diseases, Vol. 67, No. 2 (Sep.-Oct., 1940), pp. 91-96]. Consequently, individuals in the field of dentistry have struggled with the ability to eliminate bacteria from the pulpalperiapical disease process.

Decay under enamel invades the dentin tubules, and the bacteria (or bacterial byproducts or bacterial enzymes) may penetrate the pulp cavity. Bacterial invasion of the pulp cavity is common during placement of dental restorations, for example, dental fillings or crowns. Many full coverage restorations today are cemented with acid etched cements that penetrate the dentin tubules and potentially damage the pulp. However, the pulp's vitality is adaptive and osmotic pressure changes that occur inside the pulp gel may be dissipated by “self-altering” the vascular hydrostatic pressure balance to a homeostatic state. Thus, early treatment of the pulp cavity or bacterial lesions in the pulp may provide an opportunity for the pulp to repair itself.

Bacterial invasion can destroy the dentin and pulp tissue. When pulpal disease is irreversible, root canal therapy is needed to save the tooth. Currently there are several irrigation agents being used to clean and biologically “sterilize” the root canal system. Throughout the world, formaldehyde products such as formocresol continue to be used routinely in dentistry. Other agents such as citric acid, EDTA, Chlorohexidine, and NaOHCl are also used in combination with various instruments to cleanse the bacteria from the root canal system. However, these agents are not fully effective and are quite toxic (particularly NaOHCl, or bleach). Further, these toxic agents may leave residue in the root canal system when used as endodontic irrigants.

Several mechanical devices are being used together with dental irrigants to clean and sterilize the root canal. For example, the self adjusting file (SAF) is a hollow and thin cylindrical nickel-titanium lattice that adapts to the cross-section of the root canal [Metzger, et al. J Endod. 2010 Apr; 36 (4):679-90]. A single file is used throughout the root canal debridement procedure. The SAF is inserted into a path initially prepared by a #20 K-file and operated with a transline- (in-and-out) vibration. The resulting circumferential pressure allows the file's abrasive surface to gradually remove a thin uniform hard-tissue layer from the entire root canal surface, resulting in a canal with a similar cross-section but of larger dimensions. The hollow SAF file is operated with a constant flow of irrigant that enters the full length of the canal and that is activated by the vibration and is replaced continuously throughout the procedure.

Other dental irrigation devices include:

(1) laser-driven devices, such as the Waterlase MD™ Turbo with Endolase™ Radial Firing tips (Henry Schein® Dental), which uses laser energy to penetrate deep into the dentinal tubules in the root canal to reduce bacteria; and

(2) ultrasonic devices, such as Vibringe® (Amsterdam, Holland), an endodontic device for root canal irrigation, which uses a microchip controlled irrigation syringe to introduce high frequency sonic vibrations to ejected irrigant liquid.

However, none of the current instrumentation and technologies is completely effective in debridement of the root canal, particularly in preventing bacterial invasion.

When it comes to fighting off bacteria, a more holistic, less toxic, approach is often used, typically in conjunction with other antibacterial agents. For example, berberine and berbine, found in golden seal, barberry and Oregon grape, are the alkaloids responsible for the antibiotic actions of many botanicals. Berberine has been shown to increase the blood supply to the spleen and to promote the release of compounds such as tuftsin that potentiate immune function. Berberine has also been shown to activate macrophages.

Berberine has shown antimicrobial activity against bacteria, protozoa, and fungi, including: Staph sp., Strep. sp., Chlamydia sp., Corynebacterium diphtheria, E. coli, Salmonella typhi, Vibrio cholerae, Diplococcus pneumonia, Pseudomonas sp., Shigella dysenteriae, Entamoeba histolytica. Trichomonas vaginalis, Neisseria gonorrhoeae and meningitis, Treponema pallidum, Giardia lamblia, and Leishmania donovani. Berberine's action against Candida may be stronger than that of antibiotics commonly used for these pathogens. Similarly, berbine exhibits antimicrobial activity.

Another way to rid the body of microorganisms is to use plasma gas illumination, which at room temperature and pressure, has been engineered as a disinfectant to kill microorganisms, quickly inactivating not only bacteria but also viruses and fungi [Eisenberg, Anne. “Hospital-Clean Hands, Without All the Scrubbing”. NY Times. Published Feb. 13, 2010].

There have been many documented cases of plasmas being applied for sanitizing skin or other body parts. Plasma cleaners make their antibacterial cocktails by running electrical current through air. The electric current ionizes the oxygen, nitrogen and water vapor in the air, eventually creating nitric oxide, hydrogen peroxide and particles effective against bacteria.

Citation or identification of any document in this application is not an admission that such document is available as prior art to the present invention.

### SUMMARY OF THE INVENTION

The present invention is directed to methods of root canal debridement, comprising applying to the root canal a dental irrigant comprising one or more botanical agent(s).

It is an object of this invention to provide a method of root canal debridement, comprising applying to the root canal an ultrasonic irrigation needle having a continuous flow of irrigant, wherein the irrigant comprises one or more botantical agent(s).

It is also an object of this invention to provide a method of root canal debridement, comprising filing the root canal with a self adjusting file having a continuous flow of irrigant, wherein the irrigant comprises one or more botantical agent(s).

It is also an object of this invention to provide a method of root canal debridement, comprising applying to the root canal a laser device having a continuous flow of irrigant, wherein the irrigant comprises one or more botantical agent(s).

Another object of this invention is to provide a method of root canal debridement, comprising: (a) filing the root canal with a self adjusting file having a continuous flow of irrigant; and (b) applying to the root canal an ultrasonic irrigation needle having a continuous flow of irrigant, or applying to the root canal a laser device having a continuous flow of irrigant, wherein the irrigant comprises one or more botantical agent(s).

Yet another object of this invention is to provide a method of preventing tooth decay and/or microbial tooth disease, comprising applying to a dental cavity a composition comprising one or more botanical agent(s), wherein the composition is applied prior to placement of a dental restoration. The dental restoration of the present invention may be, but is not limited to, a dental tilling or crown.

In one embodiment of the invention, the botanical agent is berberine.

In one embodiment of the invention, the botanical agent is Noni.

In other embodiments of the invention, the methods may further comprise adding deionized water to a dental cavity in combination with the dental irrigant or comprised within the dental irrigant.

In other embodiments of the invention, the methods may further comprise an irrigant comprising an amino acid. In one embodiment, the amino acid may be arginine.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

The instant invention is directed to methods of using botanical-based irrigants, optionally in combination with mechanical dental debridement devices to treat or prevent microbial dental disease. In particular, the claimed invention relates to methods of using botanical-based compositions in a rinse, gel- or paste-like form to prevent microbe invasion of dental cavities, including the root canal.

Irrigant, as refered to herein, is a composition used for debridement of a dental cavity. e.g., root canal. Compositions of the present invention may comprise an irrigant and may be in the form of a gel- or paste-like material. Irrigants of the present invention preferably include one or more botanical agent(s) and/or extract(s).

Botanical or botanical agent, as referred to herein, may be used interchangeably and is an organic plant or organic plant extract or derivative.

Botanicals of the present invention may include, but are not limited to Noni (morinda citrifolia, a citric acid fruit juiced used in Polynesia as an antibacterial, antiviral and antifungal agent), alfalfa, basil, chamomile, cinnamon, clove, eucalyptus, parsley, peppennint, rhubarb, tumeric, uva ursi, yucca, buchu, chaparral, echinacea, garlic, goldenseal, myrrh, red clover, yellow dock, kava kava, kombucha, parsley, St. John's wort, skullcap, thyme and turmeric. In one embodiment, mustard seeds and their derivatives, including but not limited to indoles and polysaccharides derivatives including but not limited to d-mannose, xylitol, and glycosides including but not limited to bec5-solasonin3, solamargine, and potassium iodide may also be used in irrigants of the present invention.

In a preferred embodiment, the botanical of the present invention is goldenseal or grape extract. In another preferred embodiment, the botanical may comprise berberine alkaloid (berberine) or berbine. In other embodiments of the invention, botanical extracts, oils or derivative thereof are used in the claimed methods. In one embodiment, the botanical agent may be present in an amount of between about 0.1% and about 20% w/w of the medicament. In another embodiment, the antimicrobial agent is present in an amount of between about 0.1% and about 5.0% w/w of the medicament.

In one embodiment of the invention, the irrigant may have a pH between about 2.0 and about 11.0, although the pH may be lower or higher, depending on the choice of irrigant. In a preferred embodiment, the irrigant may have a pH between about 5.0 and about 7.0. In other embodiments of the invention, the irrigant may comprise deionized water, which may regulate the pH of the irrigant.

The term “about” as used herein, refers to an approximate 10% variation.

In one embodiment of the invention, the irrigant may further comprise one or more antimicrobial agent and/or an antiseptic agent. An antimicrobial agent may be an antibacterial agent or an antiviral agent. Antimicrobial agents of the present invention include, but are not limited to, penicillins, cephalosporins, cephamycins, carbopenems, monobactam, vancomycin, teicoplanin, macrolides, tetracyclines, aminoglycosides, chloramphenicol, sodium fusidate, sulphonamides, quinolones, and azoles. In one embodiment, the antimicrobial agent may be present in an amount of between about 0.1% and about 20% w/w of the medicament. In another embodiment, the antimicrobial agent is present in an amount of between about 0.1% and about 5.0% w/w of the medicament. In another embodiment of the invention, the antimicrobial agent is a botanical, botanical extract, oil or derivative thereof.

Antiseptic agents used in the present invention include, but are not limited to benzalkonium chloride, cetrimide, hexachlorophene, iodine compounds, mercury compounds, alcohol, hydrogen peroxide, hexamine hippurate, triclosan, cetylpyridinium chloride, and dequalinium, boric acid, and volatile oils. In another embodiment of the invention, the antiseptic agent is a botanical, botanical extract, oil or derivative thereof. In one embodiment of the invention, the antiseptic agent may be present in an amount of between about 0.1% and 20% w/w of the irrigant. In another embodiment of the invention, the antiseptic agent is present in an amount of between about 0.1% and 5.0% w/w of the irrigant.

It is to be understood that the irrigants of the present invention may comprise botanical agents, antimicrobial agents and/or antiseptic agents. In one embodiment the botanical agent may be an antimicrobial agent or an antiseptic agent. In another embodiment, the botanical agent, antimicrobial agent and/or antiseptic agent may be the irrigant.

In one embodiment of the invention, the irrigant may be used together with camphorated monochlorphenol, formocresol, eucalyptal, cresitin, NaoHCI, or any other dental irrigant.

In one embodiment of the invention, the irrigant may further comprise one or more surfactant. Surfactants of the present invention may include, but are not limited to, fluorosurfactants, ethoxylates, sulfonates, quaternary ammonium compounds, and amine oxides.

In one embodiment, the surfactant may he present in an amount of between about 0.1% and 5% w/w of the irrigant.

The invention is further described by the following numbered paragraphs:

1. A method of root canal debridement, comprising applying to the root canal a dental irrigant comprising one or more botanical agent(s).

2. A method of root canal debridement, comprising applying to the root canal an ultrasonic irrigation needle having a continuous flow of irrigant, wherein the irrigant comprises one or more botantical agent(s).

3. A method of root canal debridement, comprising filing the root canal with a self adjusting file having a continuous flow of irrigant, wherein the irrigant comprises one or more botantical agent(s).

4. A method of root canal debridement, comprising applying to the root canal a laser device having a continuous flow of irrigant, wherein the irrigant comprises one or more botantical agent(s).

5. A method of root canal debridement, comprising:

(a) filing the root canal with a self adjusting file having a continuous flow of irrigant; and

(b) applying to the root canal an ultrasonic irrigation needle having a continuous flow of irrigant,

wherein the irrigant comprises one or more botantical agents.

6. A method of root canal debridement, comprising:

(a) filing the root canal with a self adjusting file having a continuous flow of irrigant; and

(b) applying to the root canal a laser device having a continuous flow of irrigant, wherein the irrigant comprises one or more botantical agent(s).

7. A method of preventing microbial dental disease, comprising applying to a dental cavity a composition comprising one or more botanical agent(s), wherein the composition is applied prior to placement of dental restorations.

8. The method of any one of paragraphs 1-7, wherein the botanical agent is berberine.

9. The method of any one of paragraphs 1-7, wherein the irrigant comprises one or more antimicrobial agents or wherein the botanical agent is an antimicrobial agent.

10. The method of paragraph 7, wherein the dental restoration is a dental filling or crown.

11. The method of any one of paragraphs 1-10, wherein deionized water is applied to the dental cavity or root canal.

12. The method of any one of paragraphs 1-10, wherein the irrigant further comprises deionized water.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A method of root canal debridement, comprising
(a) applying to the root canal:
(i) a dental irrigant comprising one or more botanical agent(s);
(ii) an ultrasonic irrigation needle having a continuous flow of irrigant, wherein the irrigant comprises one or more botantical agent(s); or
(iii) a laser device having a continuous flow of irrigant, wherein the irrigant comprises one or more botantical agent(s);
or
(b) filing the root canal with a self adjusting file having a continuous flow of irrigant, wherein the irrigant comprises one or more botantical agent(s).

2-4. (canceled)

5. A method of root canal debridement, comprising:
(a) filing the root canal with a self adjusting file having a continuous flow of irrigant; and
(b) applying to the root canal:
(i) an ultrasonic irrigation needle having a continuous flow of irrigant, or
(ii) a laser device having a continuous flow of irrigant;
wherein the irrigant comprises one or more botantical agents.

6. (canceled)

7. A method of preventing microbial dental disease, comprising applying to a dental cavity a composition comprising one or more botanical agent(s), wherein the composition is applied prior to placement of dental restorations.

8. The method of claim 1, wherein the botanical agent is berberine.

9. The method of claim 1, wherein the irrigant comprises one or more antimicrobial agents or wherein the botanical agent is an antimicrobial agent.

10. The method of claim 7, wherein the dental restoration is a dental filling or crown.

11. The method of claim 1, wherein deionized water is applied to the dental cavity or root canal.

12. The method of claim 1, wherein the irrigant further comprises deionized water.

13. The method of claim 5, wherein the botanical agent is berberine.

14. The method of claim 5, wherein the irrigant comprises one or more antimicrobial agents or wherein the botanical agent is an antimicrobial agent.

15. The method of claim 5, wherein deionized water is applied to the dental cavity or root canal.

16. The method of claim 5, wherein the irrigant further comprises deionized water.

17. The method of claim 7, wherein the botanical agent is berberine.

18. The method of claim 7, wherein the irrigant comprises one or more antimicrobial agents or wherein the botanical agent is an antimicrobial agent.

19. The method of claim 7, wherein deionized water is applied to the dental cavity or root canal.

20. The method of claim 7, wherein the irrigant further comprises deionized water.
